(19) United States
(12) Patent Application Publication
(10) Pub. No.: US 2005/0070596 A1
(43) Pub. Date: Mar. 31, 2005

(54) Methods for treatment of inflammatory diseases using CT-3 or analogs thereof

(76) Inventors: David Baker, London (GB); Gareth Pryce, London (GB); Gavin Giovannoni, London (GB); Alan J. Thompson, London (GB)

Correspondence Address: KATTEN MUCHIN ZAVIS ROSENMAN, 525 WEST MONROE STREET, CHICAGO, IL, 60661-3693, US

(21) Appl. No.: 10/841,474

(22) Filed: May 10, 2004

Related U.S. Application Data
(60) Provisional application No. 60/469,391, filed on May 12, 2003.

(51) Int. Cl. A61K 31/353

(52) U.S. Cl. 514/454

(57) ABSTRACT

The present invention relates to non-psychoactive derivatives of tetrahydro-cannabinol, which exhibit anti-inflammatory and analgesic activities. In particular, the present invention relates to methods of administering the derivatives and pharmaceutical compositions as therapeutic agents in the treatment of pain and tissue inflammation.

This application claims priority under 35 USC Section 119(e) of U.S. Provisional Application No. 60/469,391, filed May 12, 2003 the entire disclosure of which is incorporated herein by reference in its entirety.

### FIELD OF THE INVENTION

This application relates to anti-inflammatory agents, and in particular to the use of certain cannabinoid derivatives for the treatment of inflammatory diseases such as multiple sclerosis, and to medicinal preparations containing cannabinoids.

### BACKGROUND OF THE INVENTION

Cannabinoids

Cannabis sativa, commonly known as marijuana, has been used for several years for its medicinal effects, including antipyretic and analgesic properties. Approximately 80 cannabis constituents, termed cannabinoids, naturally occur as 21 carbon atom compounds of cannabis and analogues of such compounds and their metabolites [Mechoulam, In “Marijuna Chemistry, Metabolism and Clinical effects, Academic Press, New York (1973), pages 1-99].

The major psychoactive component of marijuana is Delta-9-tetrahydrocannabinoid (THC), which has been widely studied. Studies have shown that THC affects growth, development and reproductive activity [Pharmacol Rev. 38 (1986), pages 1-18 and 151-178; Marihuana, Pharmacological Aspects of Drug Dependence, Springer Verlag (1996), pages 83-158]. Studies in mice have shown that THC suppresses antibody formation against sheep red blood cells and causes changes in cytokine production. In vitro studies, however, have shown that THC may suppress or enhance (depending on dosage) the production of various cytokines such as IL-1, IL-6 and TNFα by leukocytic cells.

Cannabidiol (CBD) is present in most cannabis preparations (hashish, marijuana, ganja) in higher concentrations than THC. Cannabidiol. was, first isolated in 1940 by Todd and Adams [J. Amer. Chem. Soc., 6,2 2194 (1940), J. Chem. Soc., 649 (1940)]. Its structure was elucidated by Mechoulam and Shvo in 1963 [Tetrahedron, 19 (1963), page 2073]. Its absolute stereochemistry was determined in 1967 [Tet. Lett., 1109-1111 (1967)]. The synthesis of cannabidiol in its racemic form and its natural form were reported in the 1960's [J. Amer. Chem. Soc., 87, 3273-3275 (1965), Helv. Chim. Acta, 50 719-723 (1967)].

Cannabidiol has no psychotropic (cannabimimetic activity) and does not bind either the brain or the peripheral receptors, CB1 and CB2 respectively [Science 169, 611-612 (1970); “Marijuana/cannabinoids: neurobiology and neurophysiology”, ed. L. Murphy and A. Bartke, CRC Press, Boca Raton, 1-33 (1992)]. Cannabidiol has, however, been observed to have anticonvulsant effects [Pharmacol, 124, 141-146 (1982)]. Cannabidiol has also been effective in animal models predictive of antipsychotic activity, and has been found to have antipsychotic effects in the, case of schizophrenia [Psychopharmacol., 104, 260-264 (1991); J. Clin. Psychiatry, 56 485-486 (1995)].

Cannabidiol has sporadically been studied for its immunomodulatory effects in vivo and in vitro. Smith et al [Proc. Soc. Exp. Bio Med. 214 (1997), pages 69-75] demonstrated that BALB/C mice injected with cannabidiol did not show significant change in the level of mRNA of IL-1, IL-6 and TNFα. At an 8 mg/kg dose of cannabidiol, the mortality of mice sublethally injected with Legionella was not affected.

Preliminary studies by Formukong et al [Inflammation, 12, 361-371 (1988)] showed that cannabidiol inhibited PBQ-induced writhing in mice when given orally at doses up to 10 mg/kg. Cannabidiol was also shown to reduce TPA-induced erythema, which is dependent upon prostaglandin release, in mice when applied topically.

In an in vitro study, Coffey et al [Biochem. Pharmacol, 52 (1996), pages 743-51] demonstrated that THC and cannabidiol inhibited nitric oxide (NO) produced by mouse peritoneal macrophages activated by LPS and IFN.gamma. Watzl et al [Drugs of Abuse, Immunity and Immunodeficiency, Plenum Press, New York, 63-70 (1991)] studies in vitro the effects of THC and cannabidiol on secretions of IL-1, IL-2, IL-6, TNFα and IFN.gamma. by human leukocytes following activation by mitrogen, They found that both cannabinoids in low concentrations increase IFN.gamma. production, whereas in high concentrations (5-24.mu.g/ml) completely blocked IFN.gamma. synthesis, and cannabidiol decreased both IL-1 and TNFα production and did not affect IL-2 secretion.

Multiple Sclerosis

Multiple sclerosis (MS) is a chronic, inflammatory disease that affects approximately 250,000 individuals in the United States. Although the clinical course may be quite variable, the most common form is manifested by relapsing neurological deficits, in particular, paralysis, sensory deficits, and visual problems.

The inflammatory process occurs primarily within the white matter of the central nervous system and is mediated by T lymphocytes, B lymphocytes, and macrophages. These cells are responsible for the demyelination of axons. The characteristic lesion in MS is called the plaque due to its macroscopic appearance.

Multiple sclerosis is thought to arise from pathogenic T cells that somehow evaded mechanisms establishing self-tolerance, and attack normal tissue. T cell reactivity to myelin basic protein may be a critical component in the development of MS. The pathogenic T cells found in lesions have restricted heterogeneity of antigen receptors (TCR). The T cells isolated from plaques show rearrangement of a restricted number of Vα and V.beta. gene segments. In addition, the TCRs display several dominant amino acid motifs in the third complementarity determining region (CDR), which is the major antigen contact site. All together, three CDR3 motifs have been identified in T cell clones known to recognize an epitope within amino acids 86-106 of myelin basic protein. These motifs were found in 44% of rearranged TCR sequences involving one particular V.beta. gene rearranged in T cells isolated from brain of two patients with MS.

A definitive treatment for MS has not been established. Historically, corticosteroids and ACTH have been used to treat MS. Basically, these drugs reduce the inflammatory response by toxicity to lymphocytes. Recovery may be hastened from acute exacerbations, but these drugs do not prevent future attacks or prevent development of additional disabilities or chronic progression of MS (Carter and Rodriguez, Mayo Clinic Proc. 64:664, 1989; Weiner and Hafler, Ann. Neurol. 23:211, 1988). In addition, the substantial side effects of steroid treatments make these drugs undesirable for long-term use.

Other toxic compounds, such as azathioprine, a purine antagonist, cyclophosphamide, and cyclosporine have been used to treat symptoms of MS. Like corticosteroid treatment, these drugs are beneficial at most for a short term and are highly toxic. Side effects include increased malignancies, leukopenias, toxic hepatitis, gastrointestinal problems, hypertension, and nephrotoxicity (Mitchell, Cont. Clin. Neurol. 77:231, 1993; Weiner and Hafler, supra). Antibody based therapies directed toward T cells, such as anti-CD4 antibodies, are currently under study for treatment of MS. However, these agents may cause deleterious side effects by immunocompromising the patient.

More recently, cytokines such as IFN-.gamma. and IFN-.beta. have been administered in attempts to alleviate the symptoms of MS. However, a pilot study involving IFN-.gamma. was terminated because 7 of 18 patients treated with this drug experienced a clinical exacerbation within one month after initiation of treatment. Moreover, there was an increase in the specific response to MBP (Weiner and Hafler, supra).

Betaseron, a modified beta interferon, has recently been approved for use in MS patients. Although Betaseron treatment showed some improvement in exacerbation rates (Paty et al., Neurology 43:662, 1993), there was no difference in the rate of clinical deterioration between treated and control groups (IFNB MS Study Group, Neurology 43:655, 1993; Paty et al., supra). Side effects were commonly observed. The most frequent of such side effects were fever (40%-58% of patients), flu-like symptoms (76% of patients), chills (46% of patients), mylagias (41% of patients), and sweating (23% of patients). In addition, injection site reactions (85%), including inflammation, pain, hypersensitivity and necrosis, were common (IFNB MS Study Group, supra; Connelly, Annals of Pharm. 28:610, 1994).

In view of the problems associated with existing treatments of MS, there is a compelling need for improved treatments which are more effective and are not associated with the afore-mentioned disadvantages. The present invention exploits the use of certain canabinoid derivatives CT-3 to effectively treat MS.

### SUMMARY OF THE INVENTION

The present invention provides pharmaceutical compositions comprising pyrazole derivatives represented by Formula (I) which compositions are useful in the treatment of a variety of diseases including, but not limited to, inflammatory diseases and multiple sclerosis.

The present invention further comprises a method for modulating CB2 receptors in a mammal, including humans, which comprises administering to a mammal in need thereof an effective amount of a compound of Formula (I), or a functional derivative thereof: 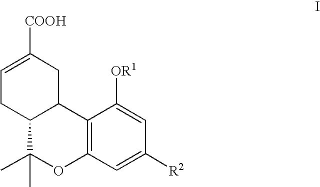
wherein R.sup.1 is a hydrogen atom, —COCH.sub.3, or —COCH.sub.2 CH.sub.3; and R.sup.2 is a branched C.sub.5-C.sub.12 alkyl, and a pharmaceutically acceptable excipient.

In one embodiment, the present invention is directed to a method of treating a mammal suffering from multiple sclerosis comprising the step of administering to the mammal a pharmaceutical composition comprising a pharmaceutically effective amount of a cannabidiol derivative compound of Formula I.

In one embodiment, the present invention is directed to a method of relieving or ameliorating the pain or symptoms associated with inflammatory diseases in a mammal suffering from multiple sclerosis comprising administering to the mammal in need thereof a therapeutically effective pain or symptom-reducing amount of a pharmaceutical composition of Formula I.

In one embodiment, the present invention is directed to a method of relieving or ameliorating the pain or symptoms associated with multiple sclerosis in a mammal suffering from multiple sclerosis comprising administering to the mammal in need thereof a therapeutically effective pain or symptom-reducing amount of a pharmaceutical composition of Formula I.

In another embodiment, the present invention is directed to a method of relieving inflammation of bodily tissue of a mammal suffering from multiple sclerosis comprising administering to the mammal in need thereof a therapeutically effective anti-inflammatory amount of a pharmaceutical composition of Formula I.

In yet another embodiment, the present invention is directed to a method of treating a mammal suffering from multiple sclerosis comprising the step of administering to the mammal a pharmaceutical composition comprising a pharmaceutically effective amount of a cannabidiol derivative compound of Formula I, wherein the cannabidiol derivative compound of the pharmaceutical composition is further combined with one or more anti-inflammatory compounds or immunomodulatory drugs.

In yet another embodiment, the present invention is directed to a method of relieving or ameliorating the pain or symptoms associated with multiple sclerosis in a mammal suffering from multiple sclerosis comprising administering to the mammal in need thereof a therapeutically effective pain or symptom-reducing amount of a pharmaceutical composition of Formula I, wherein the cannabidiol derivative compound of the pharmaceutical composition is further combined with one or more anti-inflammatory compounds or immunomodulatory drugs.

In another embodiment, the present invention is directed to a method of relieving inflammation of bodily tissue of a mammal suffering from multiple sclerosis comprising administering to the mammal in need thereof a therapeutically effective anti-inflammatory amount of a pharmaceutical composition of Formula I, wherein the cannabidiol derivative compound of the pharmaceutical composition is further combined with one or more anti-inflammatory compounds or immunomodulatory drugs.

In certain embodiments of the method of the present invention, the anti-inflammatory compound or immunomodulatory drug comprises interferon; interferon derivatives comprising betaserone, .beta.-interferon; prostane derivatives comprising iloprost, cicaprost; glucocorticoids comprising cortisol, prednisolone, methylprednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogs thereof taken either alone or in combination.

In another aspect, the present invention is directed to a method of providing neuroprotection in a mammal suffering from one or more inflammatory diseases comprising administering to the mammal in need thereof a therapeutically effective anti-inflammatory amount of a pharmaceutical composition of Formula I, wherein the amount administered is sufficient to slow the progression of disease down and/or aid in addition to symptom management.

In certain embodiments of the method of the present invention, the mammal is a human.

In certain embodiments of the method of the present invention, the cannabidiol derivative compound pharmaceutical composition is administered orally, systemically, via an implant, intravenously, topically, intrathecally, or by inhalation.

In yet another embodiment, specifically excluded from the definition of the cannabinoid derivatives contemplated for use in the methods of the present invention are those cannabinoid derivative compounds specifically disclosed in each of U.S. Pat. Nos. 6,410,588; 6,100, 259; 5,932, 610; and 5, 618,955, as if each compound were specifically recited herein.

### BRIEF DESCRIPTION OF THE DRAWINGS

FIG. 1 illustrates the structure of Δ⁹ tetrohydocannabinol superimposed on a cannabis plant. The insert shows medical grade cannabis extract (cannidor) and synthetic THC (dronabinol) pills used in cannabis trials.

FIG. 2 illustrates that the level of CB1 expression in the brain varies depending on location and is highest (intensity of green) in the basal ganglia, globus plallidus (GP) and substantia nigra (SN),with moderate levels in the cerebellum (Cer), Hippocampus (Hip) and Amygdala (Am).Low levels of expression are present in the cortex and very low levels in white matter (yellow)

FIG. 3 illustrates the structures of Endocannabinoids.

FIG. 4 illustrates the endocannabinoid agonism/degradation pathway. A membranous precursor is cleaved via the activity of a phosphodiesterase (PDE) enzyme stimulated via signals such as depolarisation, following release the endocannabinoid can either bind to the cannabinoid receptor or it is degraded through re-uptake by a diffusion facilitated transport molecule and then hyrdolyically cleaved by enzymes such as FAAH.

FIG. 5 illustrates that endocannabinoids regulate synaptic neurotransmission.

FIG. 6 illustrates that canabinoids control of neurotransmitter function.

FIG. 7 illustrates cannabinoid-mediated inhibition of spasticity in a mouse experimental model of multiple sclerosis³⁵. Compounds were injected intravenously (i.v.) and the level of THC (1 mg/kg i.v.) was matched to the THC content in a cannabis extract (obtained under UK Home Office Licence). Notably cannabis appeared to act faster than pure THC (supplied by the National Institute for Drug Abuse). It should be noted that THC has the capacity to induce maximal inhibition of spasticity. Nabilone (1 mg/kg i.v.) a synthetic analogue of THC (generously supplied by Cambridge Biomedicals, Cesamet) could inhibit spasticity to maximal levels (˜45-50% inhibition). Mean change compared to baseline (n=10/group)

FIG. 8 illustrates endocannabinoid degradation inhibitors may offer some tissue selectivity because the endocannabinoids are upregulated in areas of damage (e.g 300%). Therefore rises due to inhibition of degradation by drugs (e.g. 4 fold) will give selectivity to the lesion over that expressed in the cognitive centres which control the adverse-effects.

FIG. 9 illustrates that ajulemic acid (CT-3), a synthetic cannabinoid which dose not induce cannabimimetic effects¹⁰ inhibits spasticity (limb stiffness assessed by the force required to bend the limb) in a mouse multiple sclerosis model³⁵. ***P<0.001 compared to baseline (n<12)

### DETAILED DESCRIPTION OF THE INVENTION

As disclosed herein, the invention relates to methods of treating a variety of diseases, including, but not limited to, inflammation and multiple sclerosis in a mammal by administering a THC derivative to the mammal in need thereof an effective amount of a compound of Formula (I), or a functional derivative thereof: 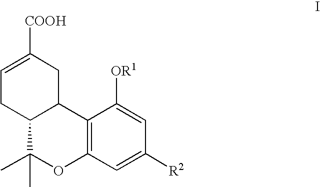
wherein R.sup.1 is a hydrogen atom, —COCH.sub.3, or —COCH.sub.2 CH.sub.3; and R.sup.2 is a branched C.sub.5-C.sub.12 alkyl.

These THC derivatives (e.g., the compounds defined by Formula I) have reduced or no psychoactivity and do not bind to the CB1 receptor. Such THC derivatives are known and can be synthesized (see, e.g., U.S. Pat. No. 5,338,753; Burstein et al., J. Medicinal Chem. 35:3185-3141, 1992; and Burstein, Pharmacol. Ther. 82:87-96, 1999).

Preferably the cannabinoid known as CT-3 or ajulemic acid or a derivative thereof is used as an anti-inflammatory agent against inflammatory diseases, especially multiple sclerosis.

The invention also provides a method of treating a patient suffering from an inflammatory disease, especially multiple sclerosis comprising the step of administering to the patient a pharmaceutically acceptable amount of cannabinoid comprising CT-3 (ajulemic acid) or a derivative thereof.

The cannabinoid comprising CT-3 (ajulemic acid) or a derivative thereof is preferably as defined above. The patient is preferably a mammal such as a human.

Cannabinoids comprising CT-3 (ajulemic acid) or derivatives thereof may be used separately or as mixtures of two or more cannabinoids. They may be combined with one or more pharmaceutically acceptable compounds such as carriers and/or excipients.

The invention also provides the use of one or more cannabinoids comprising CT-3 (ajulemic acid) or derivatives thereof as previously defined above in the manufacture of a medicament to treat inflammatory diseases, especially multiple sclerosis.

A further aspect of the invention provides a method of treating an inflammatory disease comprising the step of administering to a patient one or more cannabinoids comprising CT-3 (ajulemic acid) or a functional derivative thereof as previously defined.

In yet another aspect of the invention, specifically excluded from the definition of the cannabinoid derivatives contemplated for use in the methods of the present invention are those cannabinoid derivative compounds specifically disclosed in each of U.S. Pat. Nos. 6,410,588; 6,100, 259; 5,932, 610; and 5,618,955, as if each compound disclosed within each of U.S. Pat. Nos. 6,410,588; 6,100, 259; 5,932, 610; and 5, 618,955 were specifically recited herein.

The cannabinoid comprising CT-3 (ajulemic acid) or a functional derivative thereof may, for example, be applied orally, intramuscularly, subcutaneously, intradermally, intravenously, by nasal spray, topically, via an implant, or intrathecally.

As a general proposition, the total pharmaceutically effective amount of cannabinoid CT-3 (ajulemic acid) or a functional derivative thereof administered will be in the range of 1 ug/kg/day to 50 mg/kg/day of patient body weight, preferably 2.5 to 10 mg/kg/day especially 5 mg/kg/day.

Accordingly, the invention also relates to medicinal preparations, including topical formulations, capsules, tablets and/or injectable formulations, containing one or more cannabinoid CT-3 (ajulemic acid) or a functional derivative thereof as previously defined for use as anti-inflammatory agents.

Preferably the cannabinoid CT-3 (ajulemic acid) or a functional derivative thereof, according to any previous aspect of the invention, are used or combined with one or more known anti-inflammatory compounds. This allows advantageous properties of the cannabinoid CT-3 (ajulemic acid) or a functional derivative thereof to be combined with known properties of the known compound(s).

In the method of the present invention, one may, for example, supplement this treatment with administration of an anti-inflammatory or immunomodulatory drug. By “immunomodulatory drugs”, it is meant, e.g., agents which act on the immune system, directly or indirectly, e.g., by stimulating or suppressing a cellular activity of a cell in the immune system, e.g., T-cells, B-cells, macrophages, or other antigen presenting cells (APC), or by acting upon components outside the immune system which, in turn, stimulate, suppress, or modulate the immune system, e.g., hormones, receptor agonists or antagonists, and neurotransmitters; immunomodulators can be, e.g., immunosuppressants or immunostimulants. By “anti-inflammatory drugs”, it is meant, e.g., agents which treat inflammatory responses, i.e., a tissue reaction to injury, e.g., agents which treat the immune, vascular, or lymphatic systems.

Anti-inflammatory or immunomodulatory drug suitable for use in this invention include, but are not limited to, interferon derivatives, e.g., betaserone, .beta.-interferon; prostane derivatives, e.g., compounds disclosed in PCT/DE93/0013, e.g., iloprost, cicaprost; glucocorticoid, e.g., cortisol, prednisolone, methylprednisolone, dexamethasone; immunsuppressives, e.g., cyclosporine A, FK-506, methoxsalene, thalidomide, sulfasalazine, azathioprine, methotrexate; lipoxygenase inhibitors, e.g., zileutone, MK-886, WY-50295, SC-45662, SC-41661A, BI-L-357; leukotriene antagonists, e.g., compounds disclosed in DE 40091171 German patent application P 42 42 390.2; WO 9201675; SC-41930; SC-50605; SC-51146; LY 255283 (D. K. Herron et al., FASEB J. 2: Abstr. 4729, 1988); LY 223982 (D. M. Gapinski et al. J. Med. Chem. 33: 2798-2813, 1990); U-75302 and analogs, e.g., described by J. Morris et al., Tetrahedron Lett. 29: 143-146, 1988, C. E. Burgos et al., Tetrahedron Lett. 30: 5081-5084, 1989; B. M. Taylor et al., Prostaglandins 42: 211-224, 1991; compounds disclosed in U.S. Pat. No. 5,019,573; ONO-LB-457 and analogs, e.g., described by K. Kishikawa et al., Adv. Prostagl. Thombox. Leukotriene Res. 21: 407-410, 1990; M. Konno et al., Adv. Prostagl. Thrombox. Leukotriene Res. 21: 411-414, 1990; WF-11605 and analogs, e.g., disclosed in U.S. Pat. No. 4,963,583; compounds disclosed in WO 9118601, WO 9118879; WO 9118880, WO 9118883, antiinflammatory substances, e.g., NPC 16570, NPC 17923 described by L. Noronha-Blab. et al., Gastroenterology 102 (Suppl.): A 672, 1992; NPC 15669 and analogs described by R. M. Burch et al., Proc. Nat. Acad. Sci. USA 88: 355-359, 1991; S. Pou et al., Biochem. Pharmacol. 45: 2123-2127, 1993; peptide derivatives, e.g., ACTH and analogs; soluble TNF-receptors; TNF-antibodies; soluble receptors of interleukines, other cytokines, T-cell-proteins; antibodies against receptors of interleukines, other cytokines, T-cell-proteins; and calcipotriols and their analogues as activators of syntheses of different nerve growth factors, or these growth factors themselves or small peptides thereof which stimulate oligodendrocyte growth (or prevent their apoptosis or destruction) and enhance remyelination.

In the method of the present invention, one may, for example, supplement this treatment with administration of a calcium compound, preferably selected from calcium carbonate, calcium acetate, calcium gluconate, calcium hydrogen phosphate, calcium phosphate and calcium citrate. Preferred calcium compounds are calcium carbonate, calcium acetate and calcium citrate.

The preferred and more preferred compounds of the present invention are also similarly preferred when used in pharmaceutical compositions and for methods of treating pain associated with multiple sclerosis by administration of a compound or pharmaceutical composition according to the invention.

Treatment and Prevention of Multiple Sclerosis

As noted above, the present invention provides methods for treating and preventing multiple sclerosis by administering to the patient a therapeutically effective amount of a canabinoid CT-3 (ajulemic acid) or a derivative thereof as described herein.

The therapeutically effective amount of a canabinoid CT-3 (ajulemic acid) or a derivative thereof as described herein is thus also useful to treat the different types of MS, including the multifocal, CNS, relapsing and remitting course; the multifocal, CNS, progressive course; the single-site, relapsing and remitting course; and other variants of multiple sclerosis. See, e.g., Cecil's Textbook of Medicine, edited by James B. Wyngaarden, 1988.

The phrase “therapeutically effective amount,” means that amount of the pharmaceutical composition of the present invention that provides a therapeutic benefit in the treatment, prevention, or management of pain associated with multiple sclerosis as measured by prevention, retardation, amelioration, and/or prophylaxis of the disease.

Patients suitable for such treatment using the canabinoids of the present invention may be identified by criteria establishing a diagnosis of clinically definite MS as defined by the workshop on the diagnosis of MS (Poser et al., Ann. Neurol. 13:227, 1983). Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. Slightly lower criteria are used for a diagnosis of clinically probable MS.

Effective treatment of multiple sclerosis may be examined in several different ways. Satisfying any of the following criteria evidences effective treatment. Three main criteria are used: EDSS (extended disability status scale), appearance of exacerbations or MRI (magnetic resonance imaging).

The EDSS is a means to grade clinical impairment due to MS (Kurtzke, Neurology 33:1444, 1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step defines an effective treatment in the context of the present invention (Kurtzke, Ann. Neurol. 36:573-79, 1994).

Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are either mild, moderate, or severe according to changes in a Neurological Rating Scale (Sipe et al., Neurology 34:1368, 1984). An annual exacerbation rate and proportion of exacerbation-free patients are determined. Therapy is deemed to be effective if there is a statistically significant difference in the rate or proportion of exacerbation-free patients between the treated group and the placebo group for either of these measurements. In addition, time to first exacerbation and exacerbation duration and severity may also be measured. A measure of effectiveness as therapy in this regard is a statistically significant difference in the time to first exacerbation or duration and severity in the treated group compared to control group.

MRI can be used to measure active lesions using gadolinium-DTPA-enhanced imaging (McDonald et al. Ann. Neurol. 36:14, 1994) or the location and extent of lesions using T.sub.2-weighted techniques. Briefly, baseline MRIs are obtained. The same imaging plane and patient position are used for each subsequent study. Positioning and imaging sequences are chosen to maximize lesion detection and facilitate lesion tracing. The same positioning and imaging sequences are used on subsequent studies. The presence, location and extent of MS lesions are determined by radiologists. Areas of lesions are outlined and summed slice by slice for total lesion area. Three analyses may be done: evidence of new lesions, rate of appearance of active lesions, percentage change in lesion area (Paty et al., Neurology 43:665, 1993). Improvement due to therapy is established when there is a statistically significant improvement in an individual patient compared to baseline or in a treated group versus a placebo group.

Candidate patients for prevention may be identified by the presence of genetic factors. For example, a majority of MS patients have HLA-type DR2a and DR2b. The MS patients having genetic dispositions to MS who are suitable for treatment fall within two groups. First are patients with early disease of the relapsing remitting type. Entry criteria would include disease duration of more than one year, EDSS score of 1.0 to 3.5, exacerbation rate of more than 0.5 per year, and free of clinical exacerbations for 2 months prior to study. The second group would include people with disease progression greater than 1.0 EDSS unit/year over the past two years.

Thus, in one aspect of the present invention, the efficacy of the CT-3 cananbinoid or analogue or functional derivative thereof in the context of prevention is judged based on one or more of the following criteria: Clinical measurements include the relapse rate in one and two year intervals, and a change in EDSS, including time to progression from baseline of 1.0 unit on the EDSS which persists for six months. On a Kaplan-Meier curve, a delay in sustained progression of disability associated with MS shows efficacy. Other criteria include a change in area and volume of T2 images on MRI, and the number and volume of lesions determined by gadolinium enhanced images.

In terms of treating, preventing or ameliorating the symptoms associated with MS, the symptoms that may be treated with the pharmaceutical compositions of the present invention include one or more disabling neurological impairments such as blindness, paralysis, incoordination, and bowel or bladder dysfunction, as well as a less apparent symptom such as fatigue. As used herein “fatigue” includes loss of power, capacity to respond to stimulation, or the tiredness, or sleepiness associated with multiple sclerosis.

In another embodiment of the present invention, the one or more of the following symptoms of multiple sclerosis that may be ameliorated or prevented by treatment with the cannabidiol compounds or derivatives thereof include, but are not limited to, impairment in the following systems: pyramidal, cerebella, brainstem, sensory, bowel and bladder, visual, cerebral or other neurologic abnormality.

In another embodiment of the present invention, the one or more of the following symptoms of multiple sclerosis that may be ameliorated or prevented by treatment with the cannabidiol compounds or derivatives thereof include, but are not limited to, blocking or reducing the physiological and pathogenic deterioration associated with MS, e.g., inflammatory response in the brain and other regions of the nervous system, breakdown or disruption of the blood-brain barrier, appearance of lesions in the brain, tissue destruction, demyelination, autoimmune inflammatory response, acute or chronic inflammatory response, neuronal death, and/or neuroglia death.

In another embodiment of the present invention, the one or more of the following symptoms of multiple sclerosis that may be ameliorated or prevented by treatment with the cannabidiol compounds or derivatives thereof include, but are not limited to, preventing the disease, ameliorating symptoms of the disease, reducing the annual exacerbation rate (i.e., reducing the number of episodes per year), slowing the progression of the disease, or reducing the appearance of brain lesions (e.g., as identified by MRI scan) and postponing or preventing disability, loss of employment, hospitalization and finally death. The episodic recurrence of the mentioned diseases such as MS can be ameliorated, e.g., by decreasing the severity of the symptoms (such as the symptoms described above) associated with the, e.g., MS episode, or by lengthening the time period between the occurrence of episodes, e.g., by days, weeks, months, or years, where the episodes can be characterized by the flare-up and exacerbation of disease symptoms, or preventing or slowing the appearance of brain inflammatory lesions. See, e.g., Adams, R. D., Principles of Neurology, 1993, page 777, for a description of a neurological inflammatory lesion.

In yet another embodiment of the present invention, the time to first exacerbation and exacerbation duration and severity of any one or more of the afore-mentioned symptoms may be reduced by treatment with the cannabidiol compound or a functional derivative thereof.

According to the present invention, a pharmaceutical composition comprising an effective amount of a combination described above can be administered to patients having multiple sclerosis, e.g., multiple sclerosis variants such as Neuromyelitis Optica (Decic's Disease), Diffuse Sclerosis, Transitional Sclerosis, Acute Disseminated Encephalomyelitis, and Optic Neuritis, but also Guillain-Barre's Syndrom, virus-, bacteria- or parasite-related demylinating or otherwise degenerative brain disease such as encephalopathies related to HIV, meningococcal or toxoplasma infections, central malaria, Lyme's disease etc.

As used herein, “modulator” means both antagonist and agonist. Preferably the present modulators are antagonists.

As used herein, “treatment” of a disease includes, but is not limited to prevention, retardation and prophylaxis of the disease.

Pharmacology

The compositions of the present invention can be used in both veterinary medicine and human therapy. The magnitude of a prophylactic or therapeutic dose of the composition in the acute or chronic management of pain associated with multiple sclerosis will vary with the severity of the condition to be treated and the route of administration. The dose, and perhaps the dose frequency, will also vary according to the age, body weight, and response of the individual patient. In general, the total daily dose range of the active ingredient of this invention is generally between about 1 and 500 mg per 70 kg of body weight per day, or about 10 and 500 mg per 70 kg of body weight per day, preferably between about 50 and 250 mg per 70 kg of body weight per day, and more preferably between about 100 and 150 mg per 70 kg of body weight per day.

It is intended herein that by recitation of such specified ranges, the ranges cited also include all those amounts between the recited range. For example, in the range about 1 and 500, it is intended to encompass 2 to 499, 3-498, etc, without actually reciting each specific instance. The actual preferred amounts of the active ingredient will vary with each case, according to the species of mammal, the nature and severity of the particular affliction being treated, and the method of administration. In general, the compositions of the present invention are periodically administered to an individual patient as necessary to improve symptoms of the disease being treated. The length of time during which the compositions are administered and the total dosage will necessarily vary with each case, according to the nature and severity of the particular affliction being treated and the physical condition of the subject receiving such treatment.

It is also understood that doses within those ranges, but not explicitly stated, such as 30 mg, 50 mg, 75 mg, etc. are encompassed by the stated ranges, as are amounts slightly outside the stated range limits.

Generally, then, each daily dose is a unit dose, i.e., tablet, cachet or capsule, which contains between about 1 mg to 700 mg of the active ingredient, or pharmaceutical composition, about 10 mg to 700 mg of the active ingredient, or pharmaceutical composition, preferably about 50 mg to 250 mg, and more preferably about 100 mg to 150 mg of the active ingredient (i.e., excluding excipients and carriers). If desired, the daily dose may include two or more unit doses, i.e., tablets, cachets or capsules, to be administered each day.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know, with no more than routine experimentation, how and when to interrupt, adjust, or terminate therapy in conjunction with individual patient response.

The term “unit dose” is meant to describe a single dose, although a unit dose may be divided, if desired. Although any suitable route of administration may be employed for providing the patient with an effective dosage of the composition according to the methods of the present invention, oral administration is preferred. Suitable routes include, for example, oral, rectal, parenteral (e.g., in saline solution), intravenous, topical, transdermal, subcutaneous, intramuscular, by inhalation, and like forms of administration may be employed. Suitable dosage forms include tablets, troches, dispersions, suspensions, solutions, capsules, patches, suppositories, and the like, although oral dosage forms are preferred.

The pharmaceutical compositions used in the methods of the present invention include the active ingredients described above, and may also contain pharmaceutically acceptable carriers, excipients and the like, and optionally, other therapeutic ingredients. In one embodiment, for example, the drug is dissolved in a vegetable oil, such as olive oil or peanut oil, and, optionally, encapsulated in a gelatin capsule. For human therapy, a preferred method of administering compounds and/or pharmaceutical compositions of Formula I is orally, in the form of a gelatin capsule.

The term “pharmaceutically acceptable salt” refers to a salt prepared from pharmaceutically acceptable non-toxic acids or bases including inorganic or organic acids. Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases, for potential salt formation with the sulfate or phosphate compounds of the invention, include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine.

The compositions for use in the methods of the present invention include compositions such as suspensions, solutions and elixirs; aerosols; or carriers such as starches, sugars, microcrystalline cellulose, diluents, granulating agents, lubricants, binders, disintegrating agents, and the like, in the case of oral solid preparations (such as powders, capsules, and tablets), with the oral solid preparations being preferred over the oral liquid preparations. The most preferred oral solid preparations are capsules.

Because of their ease of administration, tablets and capsules represent the most advantageous oral dosage unit form, in which case solid pharmaceutical carriers are employed. If desired, tablets may be coated by standard aqueous or non-aqueous techniques.

In addition to the common dosage forms set out above, the compound for use in the methods of the present invention may also be administered by controlled release means and/or delivery devices such as those described in U.S. Pat. Nos. 3,845,770; 3,916,899; 3,536,809; 3,598,123; and 4,008,719, the disclosures of each of which are hereby incorporated by reference in their entirety.

Pharmaceutical compositions for use in the methods of the present invention suitable for oral administration may be presented as discrete units such as capsules, cachets, or tablets, or aerosol sprays, each containing a predetermined amount of the active ingredient, as a powder or granules, as creams, pastes, gels, or ointments, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the carrier with the active ingredient which constitutes one or more necessary ingredients. In general, the compositions are prepared by uniformly and intimately admixing in a suitable machine the active ingredient with liquid carriers or finely divided solid carriers or both, and then, if necessary, shaping the product into the desired presentation.

For example, a tablet may be prepared by compression or molding, optionally, with one or more accessory ingredients. Compressed tablets may be prepared by compressing in a suitable machine the active ingredient in a free-flowing form, such as powder or granules, optionally mixed with a binder (e.g., carboxymethylcellulose, gum arabic, gelatin), filler (e.g., lactose), adjuvant, flavoring agent, coloring agent, lubricant, inert diluent, coating material (e.g., wax or plasticizer), and a surface active or dispersing agent. Molded tablets may be made by molding, in a suitable machine, a mixture of the powdered compound moistened with an inert liquid diluent. Those skilled in the art will know, or will be able to ascertain with no more than routine experimentation, appropriate pharmacological carriers for said pharmaceutical compositions.

### EXAMPLES

The invention is further defined by reference to the following examples describing in the compositions used in the methods of the present invention, as well as their utility. The examples are representative, and they should not be construed in any way to limit the scope of the invention.

### Example 1

Summary

Research into the cannabinoid system has many similarities with that of the opioid system. In both instances studies into drug-producing plants led to the discovery of an endogenous control system that plays a fundamental role in neurobiology. Few compounds have had as much positive press from patients as those contained within the cannabinoid system. Perhaps ironically, as these claims are being investigated in such fields as MS spasticity and pain, basic research is discovering new and exciting members of this family of compounds which have hidden qualities, the most notable of which is the capacity for neuroprotection. While the neuroscientists are exploring this and other avenues, the clinicians are carrying out large randomised clinical trials of established compounds. Even if the results of these studies are not as positive as had been anticipated, it is clear that we are only just beginning to appreciate the huge therapeutic potential of this family of compounds.

Medical History of Cannabis

Cannabis has been used recreationally for millennia and is the third most commonly used drug after tobacco and alcohol with an estimated 3,000,000 frequent users within the UK alone.¹ There has also been a steady stream of medical claims throughout history that cannabis eases limb muscles, spasms, migraine and pain.² Although there are indications of medical use in the West from the 13^{th} century, it became more widely popularised in the 19^{th} century by an Irish Surgeon, W. B. O'Shaughnessy, while serving in the British Army in India. He noted anti-convulsive, analgesic, anti-anxiety and anti-emetic properties, and cannabis became more widely used for cramps, asthma and dysmenorrhea, for which it was prescribed to Queen Victoria. However, the availability of alternative synthetic compounds and the well known psychoactive effects of cannabis led to a decline in its use by the beginning of the 20^{th} century. Although cannabis was effectively banned in the USA in 1937, cannabis and tincture of cannabis remained on the British pharmacopoeia and was occasionally used until the Misuse of Drugs Act (1971) indicated there was no medical benefit and its use was outlawed. The illegality of the drug has allowed people to obtain cannabis on the black market, self-medicate and perceive benefit. This patient-led self-investigation has fuelled claims in a large variety of indications.^{3,4} In response to such claims, patient pressure and some small scale clinical studies,⁵ the UK parliment¹ felt there was sufficient evidence in certain indications, such as multiple sclerosis and pain, to warrant further investigation in large controlled trials which are now being undertaken. The current UK position is that possession and supply of cannabis is illegal and although it has recently been moved from schedule B to schedule C, this is not an endorsement that it is a safe drug but a recognition that it does not carry the same risks as other schedule B drugs, such as amphetamine and barbiturates. Should trials show acceptable benefit, the UK Government is likely to reconsider its legalisation for medical use only.

Biology of Cannabis

The acute affects of cannabis use are well recognized. It induces a “psychoactive” mild euphoric intoxication or “high” which leads to a slight impairment of psychomotor and cognitive function. In some cases cannabis can induce a variety of intensely unpleasant psychic effects including anxiety, panic, paranoia, time distortion and feelings of impending doom (known as a “whitey”) and infrequently may lead to a longer-lasting acute psychosis involving delusions and hallucinations. Frequent users may develop an amotivational syndrome. Cannabis also induces a significant increase in heart rate and a lowering of the blood pressure due to vasodilatation, causing the classic “red eye”, appetite stimulation, (known as “the munchies”), dry mouth and dizziness.¹ These may be considered as adverse effects, but all are based on basic biology, which is beginning to be uncovered.

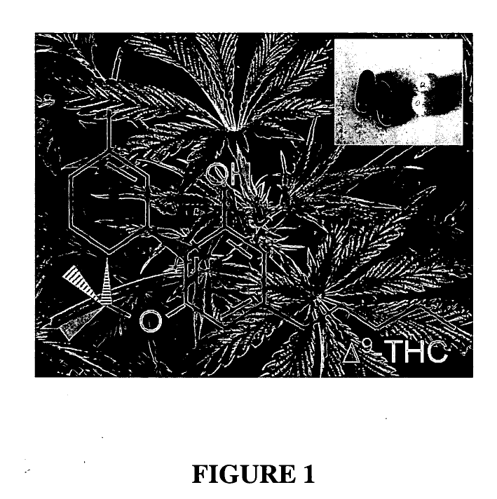

The cannabis plant (Cannabis sativa. FIG. 1) contains a large number of compounds but it was not until 1964 that the major psychoactive ingredient delta 9 tetrahydrocannabinol (THC) was discovered (FIG. 1).^{6,7} THC breaks down to produce cannabinol that was identified, along with cannabidiol, (CDB) the major non-psychoactive component, in the 1940's.^{2,7} Δ⁹THC is concentrated in the flowering head of the female plant and selective growing, in the past five to ten years has dramatically increased THC content from 1-3% THC in the “flower-power” era to 6-13% and above. Thus, the experiences of the past may be very different from the present. Cannabis may contain over 60 cannabinoid compounds and some, such as CBD, may modulate the response to THC.^{2,7,8} Understanding how these different compounds act has just only begun to become clearer in the past decade.

The Cannabinoid System

Cannabinoid Receptors

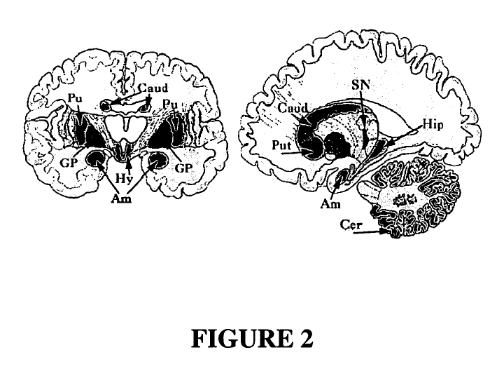

Cannabinoids are highly lipophilic, and it was originally thought that, like alcohol, the cannabinoids simply diffused through cells to mediate their functions. However, in 1990 the first cannabinoid receptor, termed CB1, was identified and cloned and this has revolutionized the field of cannabinoid biology.^{9,10} CB1 is by far the most abundant 7 transmembrane-spanning, G-protein coupled receptor in the central nervous system and is expressed on CNS neurons, as well as PNS and other peripheral cell types.¹⁰ CB1 is negatively coupled to adenylate cyclase and either negatively or positively associated with selective ion channels.^{9,11} CB1 is highly expressed in the basal ganglia, cerebellum and hippocampus and this becomes consistent with the well-known effects of cannabis on motor co-ordination and short-term memory processing, that are controlled by these brain regions (FIG. 2).¹⁰ Likewise, CB1 is expressed in the dorsal primary afferent spinal cord regions, which are known be important in pain pathways, whereas it is expressed at low levels in the brain stem,¹⁰ which control many autonomic functions. This may account for the relative lack of cannabis induced acute-fatalities.¹ Therefore the wide number of effects that cannabis can exhibit is due to the presence of CB1 in regions that control these different neurological functions. The responsiveness of the receptor is dynamic and appears to exist in a partially precoupled state that can offer different levels of stimulation in different brain regions.¹⁰

A second receptor CB2 was found and appears to be expressed primarily by leucocytes and, in contrast to CB1, does not signal ion channels.^{10,11} CB2 has no known neurological activity but it may function in haematopoetic development. There has been substantial development in the biology of CB1 and selective agonists, antagonists and mice lacking both CB1 and CB2 have been generated which can be used to understand cannabinoid biology.10,12,13 There is increasing evidence for additional “unknown” novel receptors (“CB3”) that exhibit cannabimimetic and also therapeutic effects independent of CB1 and CB2 receptors.^{10,14,15} These are more likely to be functionally rather than structurally related, as there is currently no evidence for additional CB receptors in the human genome. Furthermore, cannabinoids may also influence other receptor systems, possibly through effects on second messenger systems or through allosteric effects due to membrane insertion of cannabinoids.¹⁶ Mice that lack CB1 receptors appear remarkably normal,^{10,13} suggesting some compensatory mechanism. However, when normal homoestasis is lost, as occurs in disease, control of the cannabinoid system may be particularly important.

Endocannabinoids

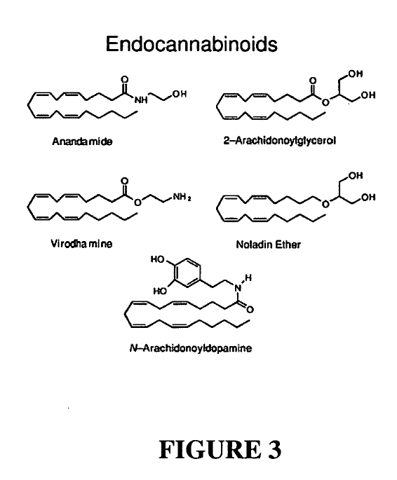

A number of endogenous “endocannabinoids” or fatty acid ligands exist. The first, discovered in 1993, was anandamide (arachidonylethanolamide) followed by 2-arachidony glycerol (2-AG. (FIG. 2)^{10,17,18} In the past two years noladin ether, virodamine (O-Arachidonlethanolamine), NADA (N-archidonyldopamine), DEA (Docosatetraenylethanolamide) have been found in the CNS. They have cannabinoid receptor binding activity, but their exact physiological relevance have yet to be described (FIG. 3).^{19,20}

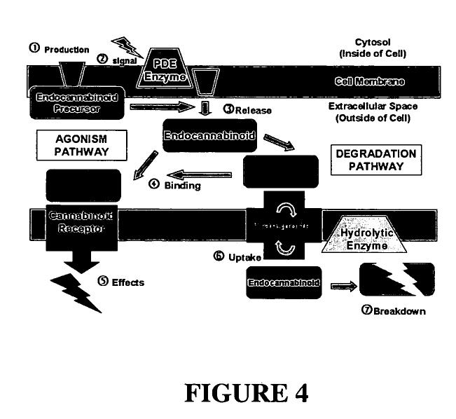

Of the endocannabinoids, the biology of anandamide and 2-archidonoyl glycerol has been the most studied.²¹ Both are produced “on demand” from membrane associated precursors by distinct biochemical pathways involving a phospholipase D and C respectively. These can then bind and stimulate the CB receptors. Anandamide and NADA can also stimulate vanilloid receptors (VR-1), which are heat gated, non-selective, ion channels associated with hyperalgesia and account for some non-CB mediated cannabimetic effects, such as the vasoactivity of anandamide on vascular beds.^{10,22} Consistent with a homoestatic role of cannabinoids, there is also a degradation system (FIG. 4) that involves re-uptake into the cell by ‘putative’ diffusion-facilitated endocannabinoid selective transporter(s) and hydrolysis by fatty acid amide hydrolase (anandamide and 2-AG) or a monoacylglycerol lipase (2-AG).^{23,24} Noladin ether is degraded by acylation.²⁰ Fatty acid amide hydrolase (FAAH) is highly expressed in the liver and often post-synaptically to CB1 and is involved in degradation of oleamide an endogenous sleep-inducing compound related to endocannabinoids.²³ This degrades anandamide to arachidonic acid and ethanolamide, which do not have CB1 binding activity.²³

Function of the Cannabinoid System

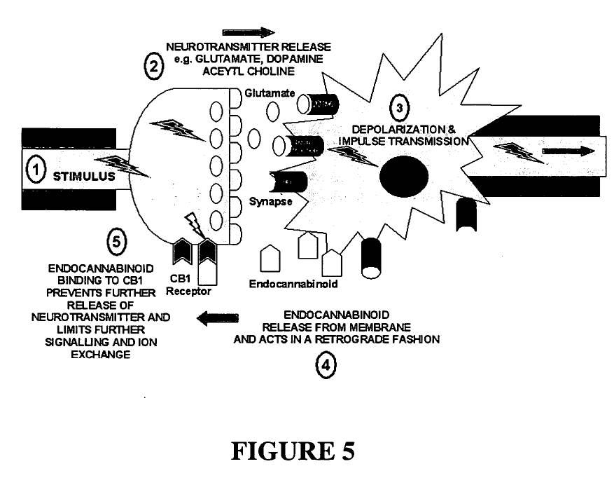

Recently it has been shown that the major function of the endocannabinoid system is to regulate synaptic neurotransmission. The CB1: endocannabinoid system regulates synaptic neurotransmission of either or both excitatory and inhibitory circuits.^{10,25} In response to depolarization and Ca²⁺ fluxes and in some instances post-synaptic group I metabotrophic glutamate receptor ligation, endocannabinoids are released which retrogradely inhibits further neurotransmitter via stimulation of pre-synaptic CB1 receptors (FIG. 5).²⁵

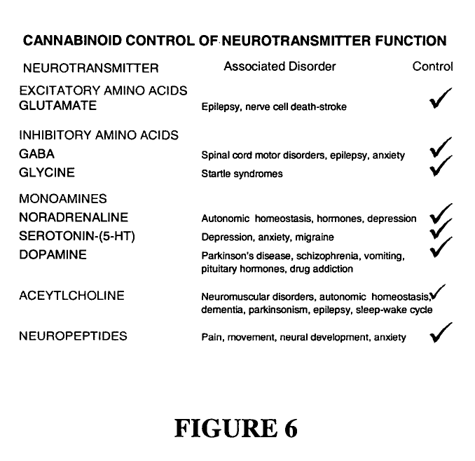

When seen as a regulator of neurotransmission,^{10,25} it is possible to envision that the cannabinoid system can influence a large number of different functions in either a positive or negative way. There is experimental evidence that cannabinoids can influence the activity of the majority of neurotransmitters (FIG. 6). What actually happens following stimulation will depend on the location of the receptor within the excitatory/inhibitory neural circuit being stimulated. This may also account for the sometimes paradoxical findings that cannabis may suppress or induce certain phenotypical signs (eg.convulsions, tremor)¹ because they are probably controlled/induced by different neuronal circuits. Many neurological diseases occur due to inappropriate neuronal signals leading to too much excitation, too little inhibition or vice versa. Dopamine activity may be inhibited by cannabinoids in motor control centres.²⁶ This can be shown clinically by the capacity of nabilone to inhibit levadopa-induced dyskinesia in Parkinson's disease.²⁷ However, in different brain regions dopamine production can be associated with reward, addiction and psychosis.

A number of studies have indicated that schizophrenics often use cannabis.²⁸ While one explanation is that they may be attempting to self-medicate excessive dopamine levels, recent evidence suggests cannabinoids may enhance dopamine release in reward centres and that juvenile and young adults smoking cannabis exhibit a small but higher risk, particularly if predisposed, to developing psychosis.^{28,29} CB1 is developmentally regulated, particularly during neural development and may be important in neuronal plasticity during foetal/post-natal periods and also at puberty and development into adulthood.³⁰ Exogenous interference in the natural brain-modelling process may have risks to behavioural development during these times and it is well accepted that chronic cannabis smoking can lead to cognitive impairment in some individuals.¹ Cannabinoids adversely affect short-term memory processing and could be disadvantageous to cognitive performance.^{1,10} However, one also has to “remember to forget” and here stimulation of the cannabinoid system may be useful to extinguish certain aversive memories such as post-traumatic fear responses.³¹ Thus cannabis may have both positive and negative outcomes and therefore its clinical use must balance these effects against the nature of the disease.

Pre-Clinical Potential of Cannabinoids—Rationale for Clinical Application

Although the clinical potential may be varied, and has prompted some to suggest that cannabis could be the “aspirin of the 21^{st} century”, much of this remains anecdotal and is too broad a scope to review in detail.^{1,3} Although there is increasing activity in understanding behavioural effects, this is hampered because of the lack of appropriate animal models with the complexity of the human brain. Therefore, experimentally, many studies have concentrated on measurable physiological effects, and there is increasing experimental understanding of the underlying biology. The vast majority of claims made by patients suggest that cannabis may be useful in symptom management^{1,3,4} and there is now experimental support for the clinical investigations of cannabis in control of pain and multiple sclerosis (MS).

Pain and Spasticity

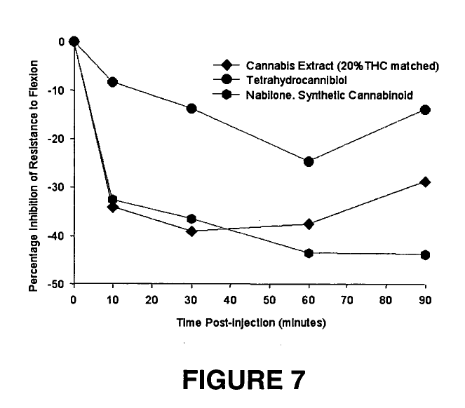

Cannabinoids inhibit pain in virtually every experimental pain paradigm either via CB1 or by a CB2-like activity either in supra spinal, spinal or peripheral sites, dependent on the type of noniception being studied.^{32,33} This is consistent with high levels of CB1 receptors on primary afferent nociceptors, particularly in the dorsal spinal cord,¹⁰ whereas peripheral CB2 receptors have been implicated in the control of inflammatory-induced pain.^{10,34} One of the major claims for cannabis in the UK is the alleviation of painful spasms and spasticity.⁴ These effects are currently difficult to assess objectively in the clinic, due to lack of sensitive and reliable outcome measures. In an experimental MS model, there is clear evidence for the tonic control of spasticity and tremor by cannabinoids.^{35,36} Although cannabis may contain more than just THC in its therapeutic armoury, the major anti-spastic activity appears to be mediated through CB1 and comparable efficacy may be obtained using single pharmacological reagents (FIG. 7). Despite early promise, there is no useful evidence to support an anti-spastic role for CB2.³⁵ Non-cannabis-derived cannabinoids can inhibit spasticity by an unknown CB1-independent mechanism.¹⁵

Although CB1 agonism can inhibit spasticity, the important experimental observation was that CB1 receptor antagonists made spasticity transiently worse^{35,36} pointing to inhibition of a tonically active, endogenous control mechanism. Indeed, inhibition of the degradation pathways of endocannabinoids by targeting the endocannabinoid transporter or FAAH degradation of the endocannabinoids led to a significant anti-spastic effect comparable to strong CB1 agonists. Importantly, such compounds do not directly bind to CB1 and thus have little inherent psychoactivity.^{35,36} Likewise, there appears to be local up-regulation of endocannabinoids in lesional areas³⁵ and therefore degradation inhibitors may offer some site selectivity not afforded by cannabinoid agonists. Similar dysregulation of the cannabinoid system is found in experimental pain³⁷ and experimental models of Huntington's and Parkinson's disease.Manipulation of the endocannabinoid system may thus offer potential in a range of neurological conditions^{38,39} including stroke.⁴⁰

Bladder Dysfunction

Bladder hyper-reflexia, a common problem in neurological disorders such as MS has been treated by local administration of VR-1 agonists.⁴¹ This symptom can be inhibited experimentally, not only by VR-1 agonists but also by cannabinoids which are considerably less irritant than VR-1 agonists.⁴² Recent work has suggested that VR-1 stimulated effects may initiate cannabinoid receptor mediated tone and could thus be a downstream effector arm of capsaicin-induced control of bladder hyper-reflexia.⁴³ As we understand more of the way in which cannabinoid receptors interact, it may be possible to use a combination of agents to limit the cannabinoid dose and thus limit the adverse effects further. However, these studies highlight one fundamental problem with cannabis as a drug: the major target for most therapeutic activities is the CB1 receptor and this is the same receptor that causes the adverse effects. It is probably going to be impossible to dissociate the adverse and therapeutic effects of cannabis, despite frequent claims in the media to the contrary.

Clinical Studies

Based on the House of Lords report (1998) that considered all available evidence,¹ it was felt that the best-supported indications were spasticity in MS and pain. Subsequently major multicentre trials of dose-titrated oral cannabis and THC (Marinol) were initiated in MS spasticity (n=660)⁴⁴ and acute, post-operative pain (n=400). Recruitment to the MS study is now complete and results are expected in June of this year. Sub-studies evaluating the effect of these agents on cognitive and bladder function in MS are also being carried out. The former is particularly important given the known effects of cannabis on cognitive function.⁴⁵ Major phase I trials of sublingual cannabis (high THC, high CBD or THC:CBD (1:1) are being undertaken in MS and other conditions including pain, sleep disturbances, and cancer-associated pain.⁴⁶ Other ongoing studies include THC hemisuccinate, administered as a suppository⁴⁷ and trials in North America are investigating the effects of cannabis smoking in MS and HIV. Therefore concerns about route of delivery are being addressed and in the near future we should know whether there is at least any scientifically demonstrable efficacy in MS and have comprehensive safety data.

As a prelude to the publication of these phase III studies, there has been a number of recent cannabis/cannabinoid trials in pain and MS spasticity which may give us a flavour of what may follow and which council against being over-optimistic. A qualitative systematic review of nine small randomised trials of cannabinoids (THC) comprising 222 patients in acute, cancer and chronic pain questioned its efficacy.⁴⁸ Where comparisons were made in acute pain studies, the level of pain was similar to current analgesics and most trials reported adverse cannabimimetic effects. However, whilst other analgesics may be able to better manage acute pain, there is a need to manage chronic (neuropathic) pain that is relatively intractable to standard analgesics. Here benefit^{1,3,4} is better supported by clinical studies.³² Oral nabilone was investigated in people with chronic pain (n=60) and of those with MS, six of 16 reported some analgesic effect. However, many experienced adverse events and opted to discontinue the drug, despite obtaining a benefit.⁴⁹. Nevertheless a phase III trial (n=96) of dose-titrated oral nabilone is currently being undertaken in neuropathic pain. More recently it has been reported in a phase II trial that sublingual cannabis (n=34) exhibits a significant analgesic effect in chronic pain in the majority (n=28) of patients with both THC-rich and, interestingly, CBD-rich cannabinoids.⁵⁰

Despite claims that smoking cannabis improves spasticity,⁴ clinical evidence for the value of cannabis has been limited to a few case reports and some small-scale studies (n=43.).⁵ A number of studies have been reported over the last 12 months. In a recent, blinded, phase I/II study (n=16) oral cannabis (THC and plant extract) failed to demonstrate efficacy in spasticity in MS and indeed both preparations worsened the patients' global impression, suggesting that at least via this route cannabis may not be the panacea.⁵¹ In a larger, dose-titrated phase II study (n=57), efficacy has been suggested in the reduction of spasms, although objective measures did not reach significance and patients were also receiving in-patient rehabilitation during the study.⁵² Although published data is not yet available, phase II studies using a sublingual spray of THC containing cannabis extracts have reported significant benefit in bladder hyper-reflexia (n=19)⁵³ and MS symptoms (n=20).⁵⁴ Recently, double blinded, phase III studies of“self-titrated” sublingual cannabis extract on neuropathic pain in MS (n=66), neuropathic pain in brachial plexus injury (n=48), chronic refractory pain and sleep disturbances in MS and other neurological conditions (n=70) have been reported to show significant pain relief and a reduction in sleep disturbance in comparison with placebo.⁵⁵ Furthermore, in an additional trial in symptoms of MS (n=160), cannabis provided a significant improvement in spasticity.⁵⁵ It is reported that symptom relief, above that given by their current standard prescription medicines, could be achieved without incurring a level of unwanted effects that would interfere with day-to-day living.⁵⁵ However these data have not yet been published. Once such trials are peer-reviewed we will be in a better situation to assess efficacy.

Future Developments in Cannabinoid Therapeutics

Novel Neurological Indications—Neuroprotection

Although the current clinical use of cannabinoids focuses on symptom management, the biology of the cannabinoid system suggests that there may be other potential benefits in the treatment of neurological disease, notably the slowing of progression in neurodegenerative disorders. Selective loss of CB1 receptors in the striatum is associated with the onset of signs in Huntington's chorea before significant axonal loss occurs both in humans and animal models,⁵⁶ suggesting that some cannabinoid regulation is lost prior to development of significant pathology. However, activation of the remaining receptors through stimulation of the endocannabinoids can limit experimental Huntington's disease.⁵⁷ Neurodegeneration is the major cause of morbidity in a number of neurological diseases such as Huntington's chorea, Parkinson's disease, Alzheimer's disease, motor neuron disease and stroke.

Neurodegenerative processes may be the fundamental reason behind progressive disease in MS, despite it being considered an inflammatory diseases.⁵⁸ Although the pathways leading to such death will be different in these disorders, it is likely that there are some similarities, such as glutamate-induced excitotoxicity and damage resulting from reactive oxygen species and toxic ion imbalances, which may make damaged or demyelinated axons particularly vulnerable to death. Cannabinoids (CB1) can regulate potentially neurodegenerative effects including inhibition of excessive glutamate production and calcium ion influx via a number of ion channels and damaging reactive oxygen species.^{10,59,60}

There is increasing experimental evidence to support a neuroprotective effect of cannabinoids in experimental models including ischaemia⁶¹ and head trauma.⁶² However, in contrast to the acute glutamate-induced insult that occurs in the penumbra during stroke ischaemia, in chronic diseases this is probably a low grade insult that may be amenable to intervention with neuroprotective agents. There is experimental evidence for activity in inflammatory-mediated neurodegeneration, including experimental MS models.⁶³ Although clinical neuroprotection is an exciting prospect, clinical data is lacking and will take time to assess. However, there is recent evidence to support the inhibition of abnormal glutamate hyperactivity. This is thought to be responsible for tics associated with Tourette's syndrome and also epilepsy. Although there is no reliable data on the use of cannabis in epilepsy,⁶⁴a small scale study has demonstrated that oral THC can inhibit tics in Tourette's syndrome.⁶⁵ While THC can mediate many of these effects experimentally, other cannabinoids may contribute to the neuroprotective effect, such as the anti-oxidant properties of CBD.^{59,60,66} A synthetic, non-CB binding cannabinoid (dexanabinol, HU211) is an NMDA receptor antagonist and phase II trials have recently reported some efficacy in head trauma.⁵⁷ The CNS is plastic and can accommodate significant nerve loss prior to the development of signs. Agents that slow this process may have considerable impact in slowing the rate of disability in chronic neurodegenerative disease.

Clinical Cannabinoid Pharmacology

In the near future results of clinical trials of oral, sublingual and even smoked cannabis will be known and a definitive answer as to whether cannabis, in the form studied, offers any therapeutic potential will be known. Once the dust has settled, researchers, clinicians and government will be in an educated position to decide the next stage forward. Whilst the immediate future may lie in plant-based medicines, once we understand the biology of the disease conditions better, the future must surely lie in pharmaceuticals, either as single agents or in combination, targeting complementary cascades. There are already indications that cannabinoids can be used in synergistic combination with opioids and benzodiazapines in pain relief,³² and through such combinations doses can be reduced with the advantage of reducing side-effects. The current clinical trials, initiated in response to patient pressure, have relied on available drugs, which had to be given orally. Whilst oral efficacy of nabilone and dronabinol for their clinical indications must be recognised, oral administration is probably the least satisfactory route for cannabis. This is due to sequestration of cannabinoids into fat from which there is slow and variable release into plasma. In addition, significant first-pass metabolism in the liver, which degrades THC, contributes to the variability of circulating levels.^{7,47,68,69} This makes dose titration more difficult and therefore increases the potential for adverse psychoactive effects. Smoking has been the route of choice for many cannabis users because this delivers a more rapid “hit” and allows more accurate dose-titration. It may also alter the chemical composition such that THC carboxylic acids are readily converted to THC by heating and or baking.⁶⁹ However, this route is not considered a viable option because of the potential for long term side effects from smoke inhalation. Better delivery vehicles and routes need to be developed for currently available and future agents. These may allow better control of side effects. One approach has been the development of a sublingual spray.⁵⁵ However, it should be possible to develop formulations and inhalers for delivery into the lungs, possibly skin patches or even the development of oral pro-drugs which become active once in the blood. “Smart” inhalers are being developed which allow metered doses that can only be dispensed by the appropriate device to limit illegal use,⁵⁵ but the best form of prohibition is to develop more effective alternatives.

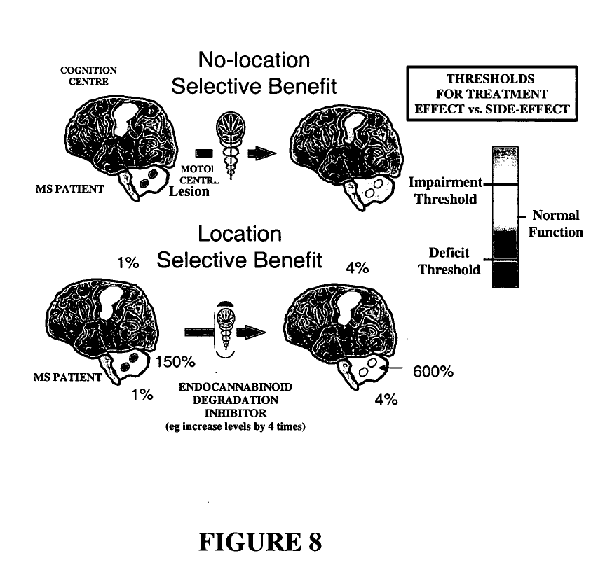

The pharmaceutical approach of making specific, potent agonists has generated some compounds that have entered preliminary clinical studies (eg. nabilone and levonantradol).⁷⁰ Likewise, CB1 antagonists (Rimonabant)⁷¹ are also currently being evaluated in the prevention of obesity. In addition, there are already hundreds of experimental agonists that could be used in future therapeutic trials. The variability of individuals to tolerate cannabinoids and the narrow window between effect and side-effect,^{7,69} however, suggest that there could be a real possibility for overdose with strong agonists. Excessive stimulation of the receptor, leads to receptor tolerization, and is a particular problem of strong agonism.⁷² Therefore the development of clinically-acceptable weak agonists may be a preferable alternative for chronic application of cannabinoid-based drugs to prevent receptor desensitization and also increase the therapeutic window. THC is only a partial CB1 agonist,¹³ whereas the endocannabinoids are weak agonists and these agents naturally stimulate receptors without much potential for inducing psychoactive effects.^{10,13} These are a new target for cannabinoid therapy.^{36,73} Endocannabinoid release could be stimulated either directly or indirectly through stimulating complementary systems (e.g metabotrophic type I glutamate receptors).²⁵ Importantly, these can also be stimulated through inhibition of endocannabinoid degradation (FIG. 4). In the case of depression, it has been found that serotonin reuptake inhibitors may be preferable to direct 5-HT receptor stimulation. This may also be the case with the cannabinoids. During disease there are alterations in endocannabinoid levels at the site of pathology.^{36,38,40} Therefore targeting endocannabinoid degradation through inhibition of the re-uptake mechanism or degradative enzymes may offer some local targeting of the cannabinoids to sites of damage whilst limiting effects in uninvolved cognitive areas [FIG. 8]. Cannabis has no mechanism to selectively target the CB1 receptor in the brain and its use will invariably be linked with some level of unwanted biological activity. Therefore the value of cannabis will depend on the extent to which the patients can titrate their dose before adverse effects become intolerable, the acceptability of which will depend on their nature and condition.

Although the brain is undoubtedly the source of many of the adverse effects, CB1 receptors are expressed on nerves outside the CNS (e.g. nerve terminals, dorsal route ganglia, vasculature).¹⁰ Selective peripheral receptor agonism may therefore, limit psychoactivity while producing benefits n areas such as pain,³³ asthma (bronchodilation),⁷⁴ and glaucoma (neuroprotection and reduction of pressure),⁷⁵ using either local application (e.g. eye drops for glaucoma) or by developing CNS-excluded agonists.

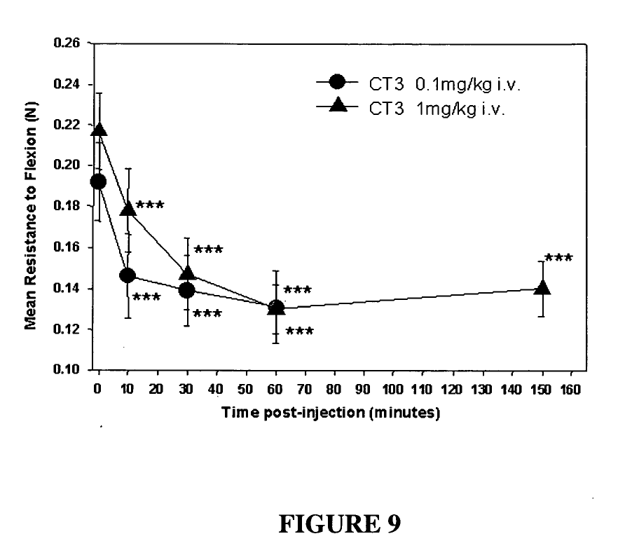

A number of experimental agents have been generated and can be shown to be as effective in experimental spasticity³¹ and pain⁶⁸ as full receptor agonists, without inducing psychoactive effects. It remains to be seen whether such agents eventually reach the clinic, and thus provide the opportunity to reap the benefits of the cannabinoid system whilst limiting the adverse effects. Ajulemic acid⁷⁶ is a cannabinoid compound that does not directly stimulate CB1 receptors to a significant extent and has undergone human safety studies and demonstrates inhibition of anandamide re-uptake and is anti-spastic, at least experimentally (FIG. 9). As we learn more about the pharmacological activities of compounds in cannabis and their biological targets outside the cannabinoid system, it may be possible to tailor varieties of cannabis to different diseases or to use a combination of known drugs which moves away from the plant into the realm of pharmaceutical medicines. Whatever the future holds there will be many challenges that need to be overcome before we view cannabinoids as routine medicine in neurological disorders.

### References

1 The United Kingdom Parliament. House of Lords. Science and Technology—Ninth Report. 1998. http://www.parliament.the-stationery office.co.uk/pa/ld199798/ldselect/ldsctech/151/15101.htm

2 Mechoulam R. The pharmacohistory of Cannabis sativa. In: Mechoulam R. (Ed). Cannabinoids as Therapeutic Agents, CRC Press, Boca Raton, 1986 pp. 119.

3 Schnelle M, Grotenhermen F, Reif M, Gorter R W. Results of a standardized survey the medical use of cannabis products in the German-speaking area. Forsch Kommplementärmed 1999; 6 (suppl.3): 28-36.

4 Consroe P, Musty R, Rein J, Tillery W, Pertwee R. The perceived effects of smoked cannabis on patients with multiple sclerosis. Eur Neurol. 1997; 38: 44-48.

5 Pertwee R G. Cannabinoids and multiple sclerosis. Pharmacol Ther 2002; 95: 165-174.

6 Mechoulam R, Gaoni Y. Hashish. IV. The isolation and structure of cannabinolic cannabidiolic and cannabigerolic acids. Tetrahedron 1965; 21: 1223-9.

7 Hawks R L. The constituents of cannabis and the disposition and metabolism of cannabinoids. NIDA Res Monogr.1982; 42: 125-37.

8 Zuardi A W, Rodrigues J A, Cunha J M. Effects of cannabidiol in animal models predictive of anti-psychotic activity. Psychopharmacology 1991; 104: 260-4.

9 Matsuda L A, Lolait S J, Brownstein M J, Young A C, Bonner T. Structure of a cannabinoid receptor and functional expression of the cloned cDNA. Nature 1990; 346: 561-4.

10 Howlett A C, Barth F, Bonner T I, Cabral G, Casellas P, Devane W A, Felder C C, Herkenham M, Mackie K, Martin B R, Mechoulam R, Pertwee R G. International Union of Pharmacology. XXVII. Classification of cannabinoid receptors. Pharmacol Rev. 2002; 54: 161-202.

11 Munro S, Thomas K L, Abu-Shaar M. Molecular characterization of a peripheral receptor for cannabinoids. Nature 1993; 365: 61-5.

12 Lutz B. Molecular biology of cannabinoid receptors. Prostaglandins Leukot Essent Fatty Acids. 2002; 66: 123-42

13 Pertwee R G. Pharmacology of cannabinoid CB1 and CB2 receptors. Pharmacol Ther 1997; 74: 129-80

14 Wiley J L, Martin B R. Cannabinoid pharmacology: implications for additional cannabinoid receptor subtypes. Chem Phys Lipids. 2002; 121: 57-63.

15 Brooks J W, Pryce G, Bisogno T, Jaggar S I, Hankey D R J, Brown P, Bridges D, Ledent C, Bifulco M, Rice A S, DiMarzo V, Baker D. Arvanil-induced inhibition of spasticity and persistent pain: evidence for therapeutic sites of action different from the vanilloid VR1 receptor and cannabinoid CB(1)/CB(2) receptors. Eur J Pharmacol. 2002; 439: 83-92.

16 Barann M, Molderings G, Bruss M, Bonisch H, Urban B W, Gothert M. Direct inhibition by cannabinoids of human 5-HT(3A) receptors: probable involvement of an allosteric modulatory site. Br J Pharmacol. 2002; 137: 589-96.

17 Vogel Z, Barg J, Levy R, Saya D, Heldman E, Mechoulam R. Anandamide, a brain endogenous compound, interacts specifically with cannabinoid receptors and inhibits adenylate cyclase. J Neurochem. 1993; 61: 352-5.

18 Mechoulam R, Ben-Shabat S, Hanus L, Ligumsky M, Kaminski N E, Schatz A R, Gopher A, Almog S, Martin B R, Compton D R, et al. Identification of an endogenous 2-monoglyceride, present in canine gut, that binds to cannabinoid receptors. Biochem Pharmacol. 1995; 50: 83-90.

19 Walker J M, Krey J F, Chu C J, Huang S M. Endocannabinoids and related fatty acid derivatives in pain modulation. Chem Phys Lipids. 2002; 121: 159-72.

20 Fezza F, Bisogno T, Minassi A, Appendino G, Mechoulam R, Di Marzo V. Noladin ether, a putative novel endocannabinoid: inactivation mechanisms and a sensitive method for its quantification in rat tissues. FEBS Lett. 2002; 513: 294-8.

21 Sugiura T, Kobayashi Y, Oka S, Waku K. Biosynthesis and degradation of anandamide and 2-arachidonoylglycerol and their possible physiological significance. Prostaglandins Leukot Essent Fatty Acids. 2002; 66: 173-192.

22 Hogestatt E D, Zygmunt P M. Cardiovascular pharmacology of anandamide. Prostaglandins Leukot Essent Fatty Acids. 2002; 66: 343-51.

23 Deutsch D G, Ueda N, Yamamoto S. The fatty acid amide hydrolase (FAAH). Prostaglandins Leukot Essent Fatty Acids. 2002; 66: 201-10.

24 Dinh T P, Carpenter D, Leslie F M, Freund T F, Katona I, Sensi S L, Kathuria S, Piomelli D. Brain monoglyceride lipase participating in endocannabinoid inactivation Proc Natl Acad Sci USA. 2002; 99: 10819-24.

25 Wilson R I, Nicoll R A. Endocannabinoid signalling in the brain. Science 2002; 296: 678-82.

26 Giuffrida A, Parsons L H, Kerr T M, Rodriguez de Fonseca F, Navarro M, Piomelli D. Dopamine activation of endogenous cannabinoid signaling in dorsal striatum. Nat Neurosci. 1999; 2: 358-63.

27 Sieradzan K A, Fox S H, Hill M, Dick J P, Crossman A R, Brotchie J M. Cannabinoids reduce levodopa-induced dyskinesia in Parkinson's disease: a pilot study. Neurology 2001; 57: 2108-11.

28 Degenhardt L, Hall W. Cannabis and psychosis. Curr Psychiatry Rep. 2002; 4: 191-6.

29 van Os J, Bak M, Hanssen M, Bijl R V, de Graaf R, Verdoux H. Cannabis use and psychosis: a longitudinal population-based study. Am J Epidemiol. 2002; 156: 319-27.

30 Kim D, Thayer S A. Cannabinoids inhibit the formation of new synapses between hippocampal neurons in culture. J Neurosci. 2001; 21: RC146: 1-5.

31 Marsicano G, Wotjak C T, Azad S C, Bisogno T, Rammes G, Cascio M G, Hermann H, Tang J, Hofmann C, Zieglgansberger W, Di Marzo V, Lutz B. The endogenous cannabinoid system controls extinction of aversive memories. Nature 2002; 418(6897): 530-4.

32 Walker J, Huang S. Cannabinoid analgesia. Pharmacol Ther. 2002; 95: 127-135.

33 Hohmann A G. Spinal and peripheral mechanisms of cannabinoid antinociception: behavioral, neurophysiological and neuroanatomical perspectives. Chem Phys Lipids 2002; 121: 173-90.

34 Calignano A, La Rana G, Giuffrida A, Piomelli D. Control of pain initiation by endogenous cannabinoids. Nature 1998; 394: 277-81.

35 Baker D, Pryce G, Croxford J L, Brown P, Pertwee R G, Huffman J W, Layward L. Cannabinoids control spasticity and tremor in a multiple sclerosis model. Nature 2002; 404: 84-87.

36 Baker D, Pryce G, Croxford J L, Brown P, Pertwee R G, Makriyannis A, Khanolkar A, Layward L, Fezza F, Bisogno T, Di Marzo V. Endocannabinoids control spasticity in a multiple sclerosis model. FASEB J 2001; 15: 300-302.

37 Walker J M, Huang S M. Endocannabinoids in pain modulation. Prostaglandins Leukot Essent Fatty Acids 2002; 66: 235-42

38 Lastres-Becker I, Fezza F, Cebeira M, Bisogno T, Ramos J A, Milone A, Fernandez-Ruiz J, Di Marzo V. Changes in endocannabinoid transmission in the basal ganglia in a rat model of Huntington's disease. Neuroreport 2001; 12: 2125-9.

39 DiMarzo V, Hill M P, Bisogno T, Crossman A R, Brotchie J M. Enhanced levels of endogenous cannabinoids in the globus pallidus are associated with a reduction in movement in an animal model of Parkinson's disease. FASEB J. 2000; 14: 1432-8.

40 Schabitz W R, Giuffrida A, Berger C, Aschoff A, Schwaninger M, Schwab S, Piomelli D. Release of fatty acid amides in a patient with hemispheric stroke: a microdialysis study. Stroke 2002; 33: 2112-4.

41 Fowler C J. Intravesical treatment of overactive bladder. =i Urology 2000; 55(Suppl 5A): 60-4.

42 Martin R S, Luong L A, Welsh N J, Eglen R M, Martin G R, MacLennan S J. Effects of cannabinoid receptor agonists on neuronally-evoked contractions of urinary bladder tissues isolated from rat, mouse, pig, dog, monkey and human. Br J Pharmacol 2000; 129: 1707-15.

43 Lever I J, Malcangio M. CB1 receptor antagonist SR141716A increases capsaicin-evoked release of Substance P from the adult mouse spinal cord. Br J Pharmacol. 2002; 135: 21-4.

44 Fox P, Thompson A, Zajicek J. A multicentre randomised controlled trial of cannabinoids in multiple sclerosis. J Neurol Sci 2001; 187: S453.

45 Bolla K I, Brown K, Eldreth D, Tate K, Cadet J L. Dose-related neurocognitive effects of marijuana use. Neurology 2002; 59: 1337-43.

46 Whittle B A, Guy G W and Robson P. Prospects for new cannabis-based prescription medicines. J Cannabis Ther 2001; 1: 183-205.

47 Mattes R D, Shaw L M, Edling-Owens J, Engelman K, Elsohly M A. Bypassing the first-pass effect for the therapeutic use of cannabinoids. Pharmacol Biochem Behav. 1993; 44: 745-7.

48 Campbell F A, Tramer M R, Carroll D, Reynolds D J, Moore R A, McQuay H J. Are cannabinoids an effective and safe treatment option in the management of pain? A qualitative systematic review. BMJ2001; 323: 13-6.

49 Notcutt W, Price M, Blossfeldt P, Chapman G. Clinical experience of the synthetic cannabinoid nabilone for chronic pain. In: Nahas G G, Sutin K M, Harvey D, Agurell S. Editors. Marihuana and Medicine, 1999. Humana Press, Totowa, pp. 567-572.

50 Notcutt W, Price M, Sansom C, Simmons S, Phillips C. Medicinal Cannabis act in Chronic Pain: Overall Results of 29 “N of 1” Studies (CBME-1). Symposium on cannabinoids, Burlington Vermont, International cannabinoid Research Society. 2002. p 55. (http://www.cannabinoidsociety.org/progab2.pdf)

51 Killestein J, Hoogervorst E L, Reif M, Kalkers N F, Van Loenen A C, Staats P G, Gorter R W, Uitdehaag B M, Polman C H. Safety, tolerability, and efficacy of orally administered cannabinoids in MS. Neurology 2002; 58: 1404-7.

52 Vaney C, Jobin P, Tscopp F, Heinzel M, Schnelle, M. Efficacy. Safety and tolerability of an orally administered cannabis extract in the treatment of spasticity in patients with multiple sclerosis. Symposium on the cannabinoids, Burlington Vt., International cannabinoid Research Society. 2002. p 57. (http://www.cannabinoidsociety.org/progab2.pdf)

53 Brady C M, Das Gupta R, Wiseman O J, Dalton C M, Berkley K J, Fowler C J. The effects of cannabis based medicinal extracts on lower urinary tract dysfunction in advanced multiple sclerosis: preliminary results. J Neurol Neurosurg Psych 2002; 72: 139.

54 Robson P J, Wade D T, Makela P M, House H. Cannabis medicinal extracts (CME), including cannabidiol, alleviated neurogenic systems in patients with multiple sclerosis and spinal cord injury. 2002 Symposium on the cannabinoids, Burlington Vt., International cannabinoid Research Society. 2002; p 56. (http://www.cannabinoidsociety.org/progab2.pdf)

55 GW Pharmaceuticals. GW announces positive results from each of four phase three clinical trials. Nov. 5, 2002. (http://www.gwharm.com/news_pres_{—}05_nov_{—}02.htm)

56 Glass M, Dragunow M, Faull R L. The pattern of neurodegeneration in Huntington's disease: a comparative study of cannabinoid, dopamine, adenosine and GABA(A) receptor alterations in the human basal ganglia in Huntington's disease. Neuroscience 2000; 97: 505-19.

57 Lastres-Becker I, Hansen H H, Berrendero F, De Miguel R, Perez-Rosado A, Manzanares J, Ramos J A, Fernandez-Ruiz J. Alleviation of motor hyperactivity and neurochemical deficits by endocannabinoid uptake inhibition in a rat model of Huntington's disease. Synapse 2002; 44: 23-35.

58 Bjartmar C, Trapp B D. Axonal and neuronal degeneration in multiple sclerosis: mechanisms and functional consequences. Curr Opin Neurol 2001; 14: 271-8.

59 Fowler C J. Plant-derived, synthetic and endogenous cannabinoids as neuroprotective agents. Non-psychoactive cannabinoids, ‘entourage’ compounds and inhibitors of N-acyl ethanolamine breakdown as therapeutic strategies to avoid pyschotropic effects. Brain Res Brain Res Rev. 2003; 41: 26-43.

60 Grundy R I. The therapeutic potential of the cannabinoids in neuroprotection. Expert Opin Investig Drugs. 2002; 11: 1365-74.

61 Nagayama T, Sinor A D, Simon R P, Chen J, Graham S H, Jin K, Greenberg D A. Cannabinoids and neuroprotection in global and focal cerebral ischemia and in neuronal cultures. J Neurosci 1999; 19: 2987-95.

62 Panikashvili D, Simeonidou C, Ben-Shabat S, Hanus L, Breuer A, Mechoulam R, Shohami E. An endogenous cannabinoid (2-AG) is neuroprotective after brain injury. Nature. 2001; 413: 527-31.

63 Pryce G, Hankey D, Ahmed Z, Baker, D. Cannanbinoid treatment and CB1 receptor involvement in inflammatory disease in the CNS 2002 Symposium on the Cannabinoids, Burlington Vt., International cannabinoid Research Society. 2002; p50. (www.cannabinoidsociety.org/progab2.pdf)

64 Consroe P. Brain cannabinoid systems as targets for the therapy of neurological disorders. Neurobiol Dis. 1998; 5: 534-51.

65 Muller-Vahl K R, Schneider U, Koblenz A, Jobges M, Kolbe H, Daldrup T, Emrich H M. Treatment of Tourette's syndrome with Delta 9-tetrahydrocannabinol (THC): a randomized crossover trial. Pharmacopsychiatry 2002; 35: 57-61.

66 Hampson A J, Grimaldi M, Lolic M, Wink D, Rosenthal R, Axelrod J. Neuroprotective antioxidants from marijuana. Ann N Y Acad Sci 2000; 899: 274-82.

67 Knoller N, Levi L, Shoshan I, Reichenthal E, Razon N, Rappaport Z H, Biegon A. Dexanabinol (HU-211) in the treatment of severe closed head injury: a randomized, placebo-controlled, phase II clinical trial. Crit Care Med. 2002; 30: 548-54.

68 Agurell S, Halldin M, Lindgren J-E, Ohlsson A., Widman M, Gillespie H, Hollister L. Pharmacokinetics and metabolism of 1-tetrahydrocannabinol and other cannabinoids with emphasis on man. Pharmacol Rev 1986; 38: 21-43.

69 Grotenhermen F. Some practice-relevant aspects of the pharmacokinetics of THC. Forch Komplementarmed. 1999; 6 (Suppl 3): 37-9.

70 Jain A K, Ryan J R, McMahon F G, Smith G. Evaluation of intramuscular levonantradol and placebo in acute postoperative pain. J Clin Pharmacol 1981; 21 suppl. 8 & 9:320S-326S.

71 Huestis M A, Gorelick D A, Heishman S J, Preston K L, Nelson R A, Moolchan E T, Frank R A. Blockade of effects of smoked marijuana by the CB1-selective cannabinoid receptor antagonist SR141716. Arch Gen Psychiatry. 2001; 58: 322-8.

72 Sim-Selley L J, Martin B R. Effect of chronic administration of R-(+)-[2,3-Dihydro-5-Methyl-3-[(morpholinyl)methyl]pyrrolo[1,2,3-de]-1,4-benzoxazinyl]-(1-napthalenyl)methanonemesylate (WIN55,212-2) or delta(9)-tetrahydrocannabinol on cannabinoid receptor adaptation in mice. J Pharmacol Exp Ther 2002; 303: 36-44.

73 Kathuria S, Gaetani S, Fegley D, Valino F, Duranti A, Tontini A, Mor M, Tarzia G, Rana G L, Calignano A, Giustino A, Tattoli M, Palmery M, Cuomo V, Piomelli D. Modulation of anxiety through blockade of anandamide hydrolysis. Nat Med. 2003; 9: 76-81.

74 Calignano A, Katona I, Desarnaud F, Giuffrida A, La Rana G, Mackie K, Freund T F, Piomelli D. Bidirectional control of airway responsiveness by endogenous cannabinoids. Nature. 2000; 408: 96-101.

75 Jarvinen T, Pate D, Laine K. Cannabinoids in the treatment of glaucoma. Pharmacol Ther 2002; 95: 203-20.

76 Burstein S H. Ajulemic acid (CT3): a potent analogue of the acid metabolites of THC. Curr Pharm Des 2000; 6: 1339-45.

77 Thompson A J, Baker D. Cannabinoids in MS; potentially useful but not just yet. Neurology 2002; 58: 1323-1324

Equivalents

The above description fully discloses the invention including preferred embodiments thereof. Modifications and improvements of the embodiments specifically disclosed herein are within the scope of the following claims. Without further elaboration it is believed that one skilled in the pharmaceutical art can, given the preceding description, utilize the present invention to its fullest extent, using no more than routine experimentation. Therefore any examples are to be construed as merely illustrative and not a limitation on the scope of the present invention in any way. The embodiments of the invention in which an exclusive property or privilege is claimed are defined as follows.

All publications, including, but not limited to, patents and patent applications cited in this specification, are herein incorporated by reference as if each individual publication were specifically and individually indicated to be incorporated by reference herein as though fully set forth.

1. A method of treating a mammal suffering from multiple sclerosis comprising the step of administering to the mammal a pharmaceutical composition comprising a pharmaceutically effective amount of a cannabidiol derivative compound of Formula (I), or a functional derivative thereof: 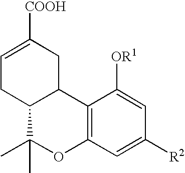
wherein R.sup.1 is a hydrogen atom, —COCH.sub.3, or —COCH.sub.2 CH.sub.3; and R.sup.2 is a branched C.sub.5-C.sub.12 alkyl, and a pharmaceutically acceptable excipient.

2. The method of claim 1, wherein R.sup.1 is hydrogen.

3. The method of claim 2, wherein R.sup.2 is a C.sub.9 alkyl.

4. The method of claim 3, wherein the C.sub.9 alkyl is a branched alkyl.

5. The method of claim 4, wherein the branched alkyl is 1,1-dimethylheptyl (CT-3 and ajulemic acid).

6. The method of claim 1, wherein R.sup.2 is a C.sub.9 alkyl.

7. The method of claim 6, wherein the C.sub.9 alkyl is a branched alkyl.

8. The method of claim 7, wherein the branched alkyl is 1,1-dimethylheptyl (CT-3 and ajulemic acid).

9. A method of relieving or ameliorating the pain or symptoms associated with multiple sclerosis in a mammal suffering from multiple sclerosis which comprises administering to the mammal in need thereof a therapeutically effective pain or symptom-reducing amount of a pharmaceutical composition according to claim 1.

10. A method of relieving or ameliorating the pain or symptoms associated with multiple sclerosis in a mammal suffering from multiple sclerosis which comprises administering to the mammal in need thereof a therapeutically effective pain or symptom-reducing amount of a pharmaceutical composition according to any one of claims 5 and 8.

11. A method of relieving inflammation of bodily tissue of a mammal suffering from multiple sclerosis which comprises administering to the mammal in need thereof a therapeutically effective anti-inflammatory amount of a pharmaceutical composition according to claim 1.

12. A method of relieving inflammation of bodily tissue of a mammal suffering from multiple sclerosis which comprises administering to the mammal in need thereof a therapeutically effective anti-inflammatory amount of a pharmaceutical composition according to any one of claims 5 and 8.

13. The method as in any one of claims 1, 5 or 8, wherein the cannabidiol derivative compound of the pharmaceutical composition is further combined with one or more anti-inflammatory compounds or immunomodulatory drugs.

14. The method of claim 13, wherein the anti-inflammatory compound or immunomodulatory drug comprises interferon; interferone derivatives comprising betaserone, .beta.-interferone; prostane derivatives comprising iloprost, cicaprost; glucocorticoids comprising cortisol, prednisolone, methylprednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof either alone or in combination.

15. The method of claim 1, wherein the mammal is a human.

16. The method of claim 1, wherein the cannabidiol derivative compound pharmaceutical composition is administered orally.

17. The method of claim 1, wherein the cannabidiol derivative compound pharmaceutical composition is administered systemically.

18. The method of claim 1, wherein the cannabidiol derivative compound pharmaceutical composition is administered via an implant.

19. The method of claim 18, wherein the implant provides slow release of the compound.

20. The method of claim 1, wherein the cannabidiol derivative compound pharmaceutical composition is administered intravenously.

21. The method of claim 1, wherein the cannabidiol derivative compound pharmaceutical composition is administered topically, intrathecally, or by inhalation.

22. The method of claim 1, wherein the amount of the cannabidiol derivative compound pharmaceutical composition administered is about 0.1 to 20 mg/kg body weight of the mammal.

23. The method of claim 21, wherein the amount of the cannabidiol derivative compound pharmaceutical composition administered is about 0.2 to 2 mg/kg body weight of the mammal.
